SEQUENCE LISTING (3 sequences)

SEQ ID NO: 1 - DNA, 16 bp, synthetic construct
  source 1..16: mol_type = other DNA
attgggtaccgttcta

SEQ ID NO: 2-3 - skipped (2 entries)

(19) United States
(12) Patent Application Publication
(10) Pub. No.: US 2026/0245661 A1
(43) Pub. Date: Aug. 20, 2026

(54) Prioritizing Candidate Cell Type-Specific Enhancers Through Comparative Genomics

(71) Applicant: Carnegie Mellon University, Pittsburgh, PA (US)

(72) Inventors: Andreas Robert Penning, Pittsburgh, PA (US); BaDoi Nguyen Phan, Pittsburgh, PA (US); Alyssa Jo Lawler, Cambridge, MA (US)

(21) Appl. No.: 19/157,192

(22) PCT Filed: Feb. 20, 2024
(86) PCT No.: PCT/US2024/016538

§ 371 Date: Aug. 15, 2025

Related U.S. Application Data
(60) Provisional application No. 63/446,625, filed on Feb. 17, 2023.

(51) Int. Cl. G16B 40/00; C12Q 1/6876

(52) U.S. Cl. CPC G16B 40/00; C12Q 1/6876; C12Q 2600/158

(57) ABSTRACT

Materials and methods for labeling and isolating particular cell types from mixed cell populations are provided herein. Also provided herein are methods for generating data representing a synthetic genetic sequence configured for labeling at least one cell type by causing expression of a marker in the at least one cell type.

### CROSS-REFERENCE TO RELATED APPLICATIONS

This application claims benefit of priority from U.S. Provisional Application Ser. No. 63/446,625, filed Feb. 17, 2023. The entire disclosure of the prior application is considered part of (and is incorporated by reference in) the disclosure of this application.

### GOVERNMENT SUPPORT CLAUSE

This invention was made with United States government support under MH130881, MH120094, DA053020, and DA046585, awarded by the National Institutes of Health. The U.S. government has certain rights in the invention

### TECHNICAL FIELD

This document relates to materials and methods for labeling and subsequently isolating particular cell types from mixed cell populations.

### BACKGROUND

The brain and other tissues consist of heterogeneous populations of cells with specialized properties and functions. Genetically and epigenetically distinguishable cell types are likely to play different roles and exhibit different responses in disease and other conditions. The vast majority of genomic assays, however, are conducted on bulk tissue and not individual cell types, making the results of such assays difficult to interpret and disentangle for cell type-specific mechanisms. Improved techniques for nuclei labeling and isolation are needed to solve this problem.

Gene therapies are a promising avenue for the treatment of a variety of diseases and disorders including pain, addiction, depression, and cancer. A major challenge in gene therapy approaches have been off target effects, especially in developing circuit-based strategies for brain disorders. Even a small piece of neural tissue can contain dozens of molecularly distinct cell types, each with their own role in a variety of neural circuits and behaviors. The ability to selectively activate or inhibit specific populations of cells would allow the implicated neural circuit to be targeted, without disrupting other key functions of that brain region.

### SUMMARY

Disclosed herein are systems and methods configured for prioritizing candidate cell type-specific enhancers through comparative genomics. Comparative genomics includes a process wherein data describing genes across many species, such as chromatin data, are analyzed to determine a relationship between genetic sequences and cell-types. These data can be used to label specific cell types with their associated sequences for gene therapy. By analyzing the relationships between genetic sequences and cell-types across many species at the same time, a sequence-cell type relationship that is conserved (or present) across multiple species can be prioritized for additional analysis. The additional analysis can validate the relationship by testing individual enhancers that are predicted with high confidence (e.g., exceeding a threshold) to label a given cell-type.

Specifically, conservation across species of cell type-specific epigenomic features that are indicative of enhancers can be used to prioritize sequences to drive cell type-specific gene expression. Conservation of predicted cell type-specific epigenetics across a larger number of species can be used to prioritize sequences to drive cell type-specific gene expression.

The prioritized sequences can be further analyzed to determine trends in these sequences. As described herein, a second machine learning model, such as a generative adversarial network (GAN), can determine which features of these sequences are of primary importance for functionality and generate efficacy predictions for synthetic sequences.

A machine learning-based approach called Specific Nuclear-Anchored Independent Labeling (SNAIL) that uses the cell type specificity of regulatory elements (RE) to control gene expression in specific neuronal cell types in the brain has been demonstrated. The system can execute one or more deep neural networks to identify candidate RE sequences with highly specific activity in a neuronal cell type of interest.

The results from the data processing system can guide a user to synthesize candidate REs and package them into an engineered adeno-associated virus (AAV) along with a transgene to express in the target cells. Transgenes can be a reporter gene, such as GFP, to validate experiments. SNAIL can also work with other transgenes, including ones that control neuronal firing or ones that report neuronal activation.

In an example, the SNAIL method is used to prioritize candidate enhancers in the striatum and cortex using Rhesus Macaque single nucleus ATAC-Seq. There is evidence of specificity for three additional cell types: Striosome rnedium spiny neurons (validated in mouse), D1 matrix medium spiny neurons (validated in mouse), and cortical layer III pyramidal neuron (validated in mouse and macaque). This machine learning technology may be used to prioritize enhancers to target neuron subtypes of the dorsal horn of the spinal cord.

In an aspect, a process is performed for interpreting genetic variants of complex neurological disorder traits with machine learning models inferring conserved functional genomes of caudate cell types across species. In some implementations, the process is performed by a data processing system including one or more processors. The process includes obtaining genetic sequence data for a plurality of species. The process includes accessing a machine learning model trained to generate, for a given genetic sequence, a prediction of an activity level for a given cell type. The process includes executing the machine learning model on a set of genetic sequences across the plurality of species for at least one cell type. The process includes, based on the executing, determining, for the genetic sequence, a predicted activity value for each cell type for each of the plurality of species to generate a set of predicted activity values. The process includes measuring, from the set of predicted values, a selective pressure of a particular genetic sequence across at least two species, the selective pressure representing an active effect for a particular cell type across the at least two species. The process includes generating, for the particular cell type, a label that associates the particular genetic sequence with the particular cell type.

In some implementations, the process includes testing the particular gene sequence to validate the predicted activity value of the genetic sequence for the particular cell type for a species of the at least two species. In some implementations, the testing includes introducing a gene sequence into a genome for the particular cell type; and measuring a gene expression for the particular cell type.

In some implementations, the machine learning model is trained by performing operations comprising training the machine learning model with input data labeled by features of a given genetic sequence correlating to a similar enhancer behavior for a cell type across two or more types of species; training the machine learning model with input data labeled by features of the given genetic sequence correlating to different enhancer behavior for the cell type across the two or more types of species; and training the machine learning model with input data where the labels are inferred across orthologous genome sequences and cell populations.

In some implementations, the features comprise identified sets of one or more nucleotides in the genetic sequence.

In some implementations, the process includes determining a set of gene sequences that are associated with the particular cell type; and training, using the set of gene sequences, a second machine learning model to generate a given synthetic gene sequence as an enhancer sequence for the particular cell type.

In some implementations, the process includes generating, using the second machine learning model, a synthetic gene sequence that acts as an enhancer sequence for the particular cell type.

In some implementations, a first species of the plurality of species is a human species, and wherein a second species of the plurality is a rodent species.

In some implementations, the genetic sequence includes an enhancer sequence.

In some implementations, the genetic sequence includes an orthologous sequence across the at least two species.

In some implementations, the genetic sequence data comprises chromatin data.

In some implementations, the plurality of species includes at least 100 species types, and wherein the at least one cell type comprises at least 10 cell types.

The systems and methods described herein enable one or more of the following advantages.

The systems and methods described herein improve enhancer design and prioritization. The systems and methods prioritize candidate cell type-specific enhancers through comparative genomics improve a screening relative to machine learning strategies that prioritize candidate enhancers as drivers of cell type-specific gene expression. The improved enhancer design enables the ability to identify genetic sequences that have an ability to target specific cell-types (e.g., neuron subtypes for the striatum and cortex example previously described).

The comparative genomics of the systems and processes described herein use chromatin data from across many species in a comparative analysis for identifying relationships to cell types. Generally, features of the genome that are more conserved are more likely to be functionally important for the fitness of the organism. Candidate enhancers that are conserved in their cell type-specificity are more likely functional in the context of driving cell type-specific gene expression.

A machine learning model is configured to predict relationships between cell types and gene sequences based on training using a small number of species and available chromatin data. The machine learning model is configured to make predictions across orthologous sequences across multiple mammals. The machine learning model can perform a selective pressure analysis by making cell-type specific predictions across hundreds of mammalian genomes. The machine learning model can generate predictions in two ways. The data processing system generates a prediction of a relationship between a cell-type and a sequence specifically, and a prediction of which sequences translate better across species to be open chromatin for a specific cell type (e.g., which sequences are labels for specific cell-types).

The machine learning models described herein have an improved accuracy for generating these predictions in comparison with existing models. The improved accuracy is enabled by training the machine learning model based on gene sequence features that are consistent labels for cell types across different species and also training the machine learning model based on features that are found to not be consistent predictors across species. For testing the model, the machine learning model is tested separately on different species, including testing both positive and negative predictive outcomes from the training data for a given cell type.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1B show an example process for interpreting genetic variants of complex neurological disorder traits with machine learning models inferring conserved functional genomes of caudate cell types across species.

FIG. 2 shows an example of a workflow that uses a sequence-based machine learning model.

FIG. 3A shows an example process for executing a machine learning model for generating synthetic genetic sequences.

FIG. 3B shows an illustration of an example a machine learning model for generating synthetic genetic sequences.

FIGS. 4A-4D show examples of pairwise human-model species caudate cell type OCR comparisons that demonstrate complex traits enriched within conserved open chromatin in the human genome.

FIGS. 5A-5B show a process for annotating cell type eQTLs and fine-mapped SNPs with Cell-TACIT Age estimates conserved cell type regulatory activity across mammalian species.

FIG. 6 shows example data of an experimental validation of candidate enhancers in a reporter assay.

FIG. 7 shows an example process for interpreting genetic variants of complex neurological disorder traits with machine learning models inferring conserved functional genomes of caudate cell types across species.

FIG. 8 shows an example computing system.

### DETAILED DESCRIPTION

The data processing system thus uses machine learning models (convolutional neural networks, deep neural networks, etc.) to prioritize cross-species predictions and to apply those predictions to prioritize enhancers for cell-type specificity. The data processing system measures selective pressure on a genome using machine learning rather than testing individual nucleotides in sequence. This approach produces more accurate predictions of relationships between gene sequences and cell types. The sequences can be validated in another testing process. As previously stated, these sequences can be used to train a GAN to design new, synthetic sequences likely to have the properties identified from the predictions of the data processing system.

In some embodiments, inputs may include epigenetic features which are markers of cell type or tissue-specific enhancer activity In some embodiments, inputs may include histone modifications. In some embodiments, inputs may include cell type-specific high-throughput reporter assays.

In some embodiments, cell type specificity may be prioritized by conservation of measured cell type-specific open chromatin across species. In some embodiments, candidate enhancers may be prioritized based on the conservation of their cell type-specific open chromatin across species. In some embodiments, conservation across species of cell type-specific epigenomic features indicative of enhancers may be used to prioritize sequences to drive cell type-specific gene expression.

In some embodiments, cell type specificity may be prioritized by conservation of predicted cell type-specific open chromatin across species. In some embodiments, candidate enhancers may be prioritized based on conservation of predicted open chromatin across a large number of species. In some embodiments, conservation of predicted cell type-specific epigenetics across a larger number of species may be used to prioritize sequences to drive cell type-specific gene expression.

In some embodiments, cell type specific enhancers may be used in concert with Designer Receptors Exclusively Activated by Designer Drugs (DREADDs), to selectively activate or inhibit neuron subtypes underlying a variety of neurological and psychiatric disorders including Parkinson's and major depressive disorder.

In some embodiments, cell type specific enhancers may be used in concert with optogenetic, light-sensitive enzymes, channels, and transcription factors that allow precise control of a neuron subtype's biochemical signaling pathway.

In some embodiments, cell type specific enhancers may be used in concert with genetically encoded calcium indicators (GECI), to selectively measure neuron subtype electrical activation to study a variety of neurological and psychiatric disorders including Parkinson's and major depressive disorder.

As previously described, the systems and methods herein are based on a development of compositions and methods for labeling and isolating specific populations of cells. The methods include SNAIL and cSNAIL (Cre-Specific Nuclear-Anchored Independent Labeling). cSNAIL and SNAIL provide improvements over current methods for nuclear isolation methods, as they are easier, more time-efficient, and more cost-effective, without any loss of selection. Moreover, cSNAIL and SNAIL have the added benefits of being compatible with multiplexing and other transgenic models, extending to new cell types, and being transferrable across species. The availability of this technology increases the practicality of cell type-specific genomics and allows these approaches to be used in other mammals, including humans. SNAIL and cSNAIL are described in greater detail in WO Pub. 2020/257516, WO Pub. 2020/257520, and Irene M. Kaplow et al, “Relating enhancer genetic variation across mammals to complex phenotypes using machine learning,” Science 380, eabm7993 (2023), the contents of each of which are hereby incorporated by reference in entirety.

Enhancers include deoxyribonucleic acid (DNA)-regulatory elements that activate transcription of a gene or genes to higher levels than would be the case in their absence. Genome profiling has revealed that general transcription factors (GTFs) and ribonucleic acid (RNA) polymerase II (Pol II) are recruited to enhancers. Enhancers serve as centers for the assembly of the pre-initiation complex (PIC).

Chromatin data refers to data describing a mixture of DNA and proteins that form the chromosomes found in the cells of humans and other higher organisms. Chromatin packages DNA into a unit capable of fitting within the tight space of a nucleus. There are two forms of chromatin-euchromatin and heterochromatin. Euchromatin consists of DNA segments involved in replication and transcription.

Epigenomics includes determining heritable marks that are materialized by chemical modification of the nucleotide bases and histones. Histones are a major type of chromosomal proteins, which are compactly wrapped around by genomic DNAs to form chromatins, to reduce chromosomal volume and strengthen the structure. The chemical modifications alongside the DNA sequences carry heritable information, passed down from cells to their offspring, by the completion of either mitotic or meiotic cell cycles. Modifications may affect the binding of transcription factors to the transcription elements, thereby changing the expression of genes.

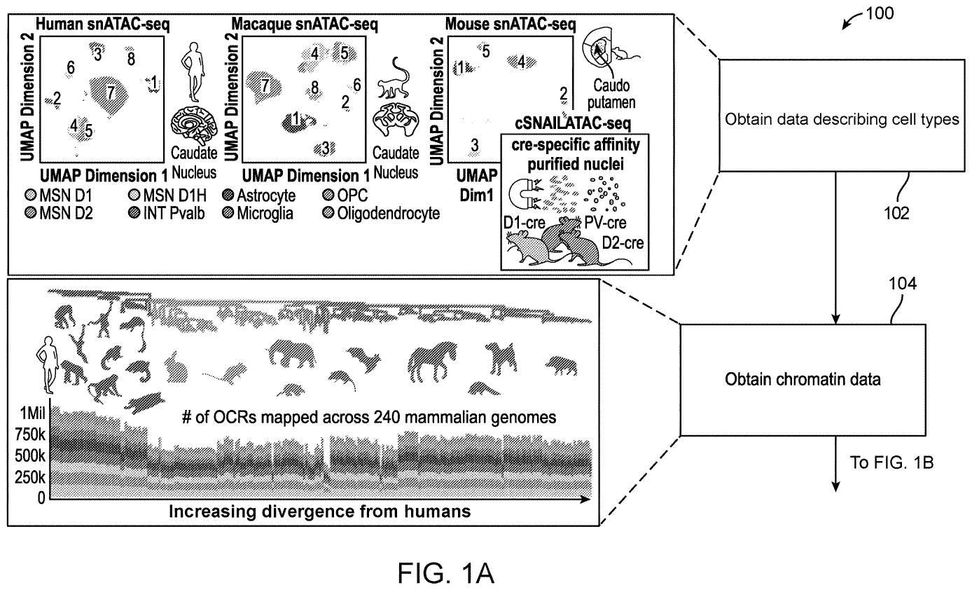

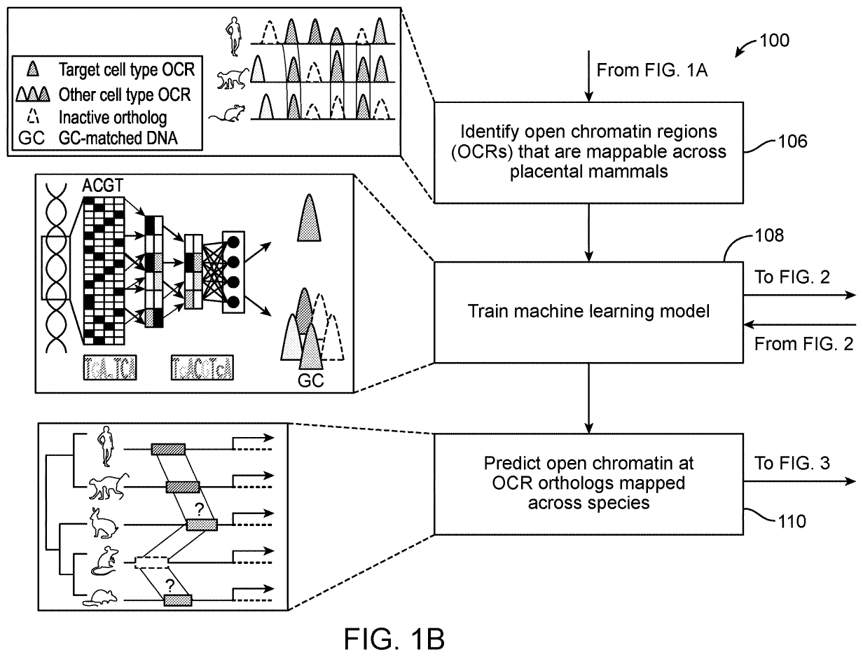

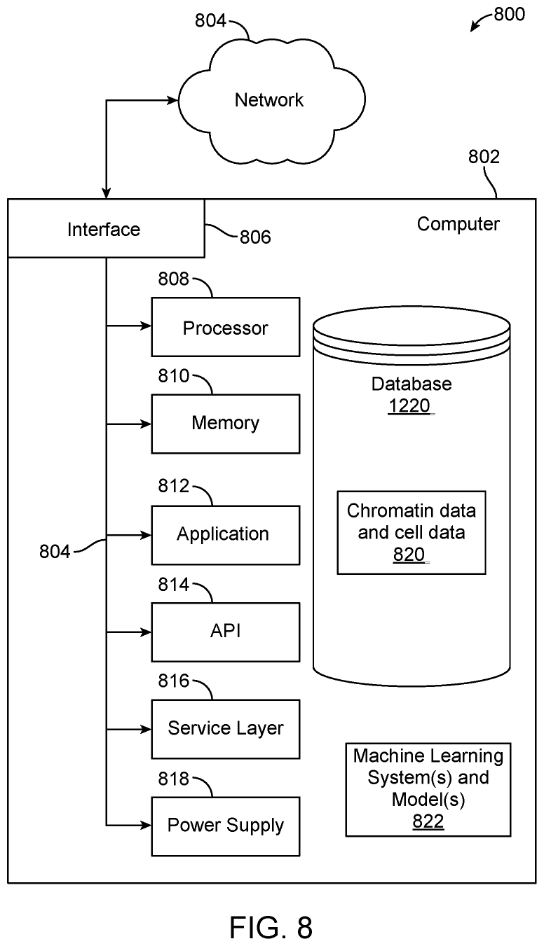

FIGS. 1A-1B show an example process 100 for interpreting genetic variants of complex neurological disorder traits with machine learning models inferring conserved functional genomes of caudate cell types across species. In some implementations, the process 100 is performed by a data processing system (such as computing system 800 of FIG. 8) including one or more processors. The computing system 800 is also called a data processing system 800 herein.

The process 100 includes obtaining (102) data describing cell types and obtaining (104) chromatin data. A data processing system executing process 100 can receive as input data any epigenetic feature that is a marker of cell type or tissue-specific enhancer activity, including histone modifications or cell type-specific high-throughput reporter assays. The process 100 generates predicts cross-species by applying the approach to cell type-specific open chromatin data (e.g., data 822) across human, macaque, mouse, and rat measured by single nucleus ATAC-Seq.

In some implementations, the process 100 includes two processes for prioritizing cell type-specificity. The process 100 uses a conservation of measured cell type-specific open chromatin across species. The process 100 uses conservation of predicted cell type-specific open chromatin across species. These approaches are subsequently described in additional detail.

The process 100 includes identifying (106) open chromatin regions (OCRs) that are mappable across placental mammals. The process 100 includes prioritizing candidate enhancers based on the conservation of their cell type-specific open chromatin across species. The data processing system identifies enhancers that drive gene expression across species in select populations of cells from a tissue using the open chromatin regions (OCRs) from across multiple species, such as the CACTUS reference-free multiple sequence alignment of 241 mammals.

For each species, the data processing system identifies pseudobulk peaks for each cell type using a standard method for the analysis of single nucleus ATAC-Seq. These steps include mapping the single nucleus ATAC-Seq peaks to the genome, clustering or labeling individual cells based on their cell type, aggregating all reads from any cell within that population, and then calling peaks on those aggregated reads.

The data processing system uses a set of multiple sequence alignment tools to map orthologous enhancer regions. For example, the data processing system can use a reference-free multiple sequence alignment, which is robust to chromosomal evolution events such as indels and inversions. To use the CACTUS alignment to identify orthologous sequences, a custom tool was created. Alone, this custom tool produces highly fragmented outputs. To extend the functionality of these resources for comparative epigenomics, a second tool, HALPER, is executed that uses a CACTUS multiple sequence alignment in combination with a halLiftover output to generate consensus orthologous regulatory elements. The output maps regions directly surrounding the peak summit center of the open chromatin to report 1-1 OCR orthologs between species.

The data processing system can identify orthologous regions for the OCRs identified in cell types for human, macaque, and mouse genes. When trying to identify a label for specific cell subtype, the data processing system prioritizes candidate enhancers where the OCR is conserved in its cell type-specificity across species.

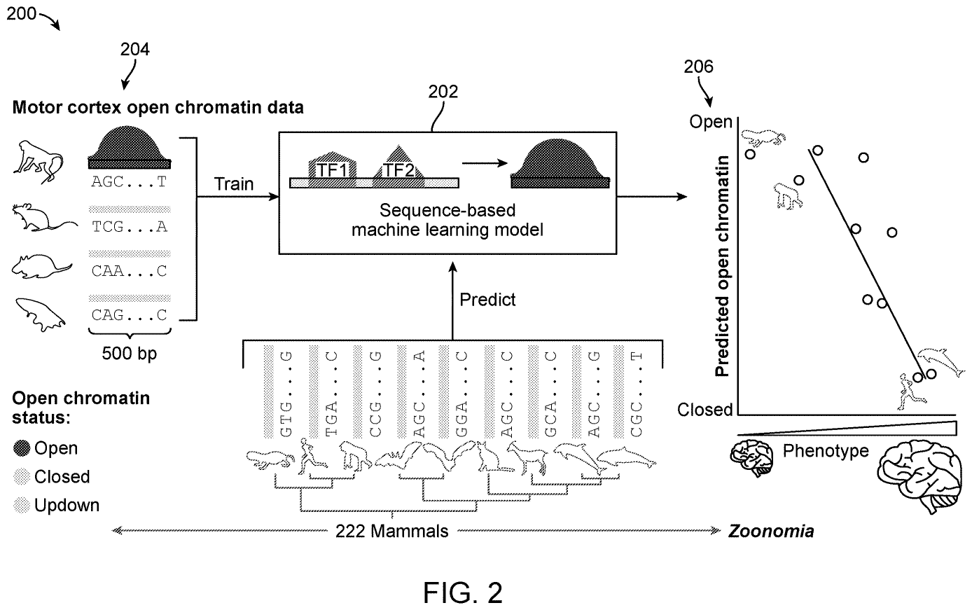

The data processing system is configured, in process 100, to train (108) a machine learning model, called Tissue-Aware Conservation Inference Toolkit (TACIT), described in further detail in relation to FIG. 2. The output can be used to predict (110) open chromatin at OCR orthologs mapped across species. In some implementations, training the machine learning model is based on input data where the labels are inferred across orthologous genome sequences and cell populations.

The data processing system prioritizes candidate enhancers based on the conservation of predicted open chromatin across a large number of species. The data processing system identifies enhancers that drive gene expression across species in select populations of cells by combining previously published machine learning results with comparative genomics research. Candidate enhancers are prioritized using machine learning models that can learn the genome sequence code underlying cell type-specific open chromatin and expression. The sequence features that are learned by the machine learning models may not necessarily be functional. Evolutionary conservation can be a powerful tool for identifying likely functional regulatory elements. Rather than looking directly at orthologous open chromatin, the conservation of the cell type-specific regulatory code across large numbers of species could be used to prioritize candidate enhancer sequences. A candidate enhancer will be prioritized if its cell type-specific regulatory code is conserved across multiple species.

The data processing system can predict a tissue-specific regulatory code across mammals. For example, the process 100 has be performed across mammals to prioritize candidate enhancers to label specific cell populations in Eurarchontaglires. The enhancer prediction 100 is performed across the genomes of 222 Boreoeutheria for motor cortex tissue and in PV+ neurons to identify signatures of convergent evolution.

In some implementations, interpreting genetic variants of complex neurological disorder traits with machine learning models inferring conserved functional genomes of caudate cell types across species. Eight cell types are identified across the primate caudate nucleus and mouse caudoputamen. A reduced dimensionality UMAP projection plots of single nucleus assay for transposase-accessible open chromatin (snATAC-seq) from rhesus macaque is performed alongside reprocessed snATAC-seq data from human, and mouse. The process 100 includes Cre-dependent Sun1GFP Nuclear-Anchored Independent Labeling (cSNAIL) ATAC-seq conducted in transgenic mouse strains.

The inferred molecular Zoonomia phylogeny, shown in relation to step 104, is aligned to a stacked histogram showing the number of human caudate cell type open chromatin regions (OCRs) that are mappable across placental mammals. The phylogenetic tree is rooted with Homo sapiens as the outgroup. Tree branches are colored by the species membership in distinct grand orders and magna orders with representative animal silhouettes.

The process 100 includes execution of a cell type-aware conservation inference toolkit (Cell-TACIT) pipeline to build convolutional neural network (CNN) models of cell type-specific regulatory activity using DNA sequence underlying OCRs and use models to predict open chromatin at OCR orthologs mapped across species. The model is described further in relation to FIG. 2.

FIG. 2 shows an example of a workflow 200 that uses a sequence-based machine learning model 202. This model is called a Tissue-Aware Conservation Inference Toolkit (TACIT). The process 200 includes generating open chromatin data 204 from a few species in a tissue related to a phenotype, using the sequences underlying open and closed chromatin regions to train the machine learning model 202 for predicting tissue-specific open chromatin and associating open chromatin predictions 206 across dozens of mammals with the phenotype.

Protein-coding sequence differences have failed to fully explain the evolution of multiple mammalian phenotypes. These phenotypes have evolved at least in part through changes in gene expression, meaning that their differences across species may be caused by differences in genome sequence at enhancer regions that control gene expression in specific tissues and cell types. Sequence conservation-based approaches for identifying such enhancers are limited because enhancer activity can be conserved even when the individual nucleotides within the sequence are poorly conserved. This is due to an overwhelming number of cases where nucleotides turn over at a high rate, but a similar combination of transcription factor binding sites and other sequence features can be maintained across millions of years of evolution, allowing the function of the enhancer to be conserved in a particular cell type or tissue. Experimentally measuring the function of orthologous enhancers across dozens of species is currently infeasible, but new machine learning methods make it possible to make reliable sequence-based predictions of enhancer function across species in specific tissues and cell types.

To overcome the limits of studying individual nucleotides, the machine learning model 202 is used in the process 200. Rather than measuring the extent to which individual nucleotides are conserved across a region, the process 200 uses a machine learning model 202 to test whether the function of a given part of the genome is likely to be conserved. More specifically, convolutional neural networks (CNNs) learn the tissue- or cell type-specific regulatory code connecting genome sequence to enhancer activity using candidate enhancers identified from only a few species. This approach allows enables a data processing system to accurately associate differences between species in tissue or cell type-specific enhancer activity with genome sequence differences at enhancer orthologs.

The data processing system connects the generate predictions 206 of enhancer function to phenotypes across hundreds of mammals in a way that accounts for species' phylogenetic relatedness. The machine learning model 202 can identify candidate enhancers from motor cortex and parvalbumin neuron open chromatin data that are associated with brain size relative to body size, solitary living, and vocal learning across a large number (e.g., 222) of mammals. The results specify multiple candidate enhancers associated with brain size relative to body size, several of which are located in linear or three-dimensional proximity to genes whose protein-coding mutations have been implicated in microcephaly or macrocephaly in humans. The data processing system, through process 200, identified candidate enhancers associated with the evolution of solitary living near a gene implicated in separation anxiety and other enhancers associated with the evolution of vocal learning ability. The data processing system generates distinct results for bulk motor cortex and parvalbumin neurons, demonstrating the value in applying the process 200 to both bulk tissue and specific minority cell type populations data. The process 200, in an example, predicted enhancer activity of over 400,000 candidate enhancers in each of 222 mammals and their associations with the phenotypes we investigated.

The process 200 leverages predicted enhancer activity conservation rather than nucleotide-level conservation to connect genetic sequence differences between species to phenotypes across large numbers of mammals. The process 200 can be applied to any phenotype with enhancer activity data available from at least a few species in a relevant tissue or cell type and a whole-genome alignment available across dozens of species with substantial phenotypic variation. Though the process 200 is applied initially to transcriptional enhancers, the process 200 can be applied to genomic regions involved in other components of gene regulation, such as promoters and splicing enhancers and silencers.

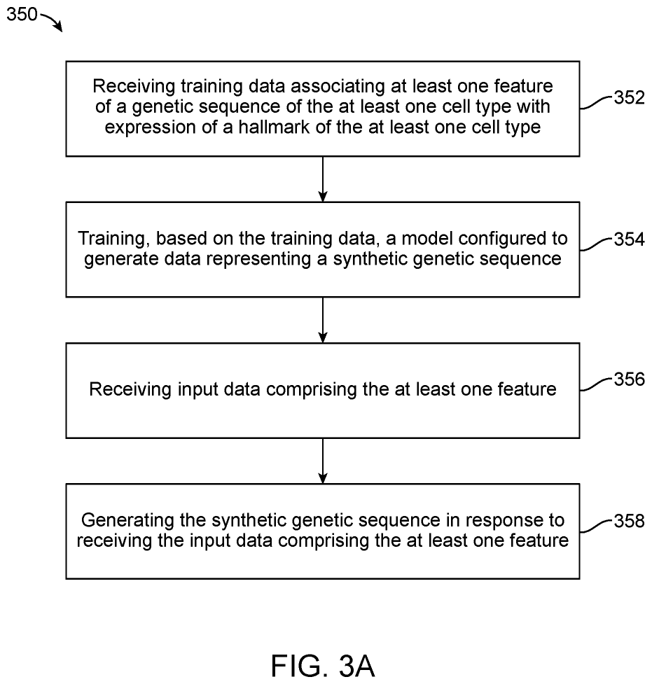

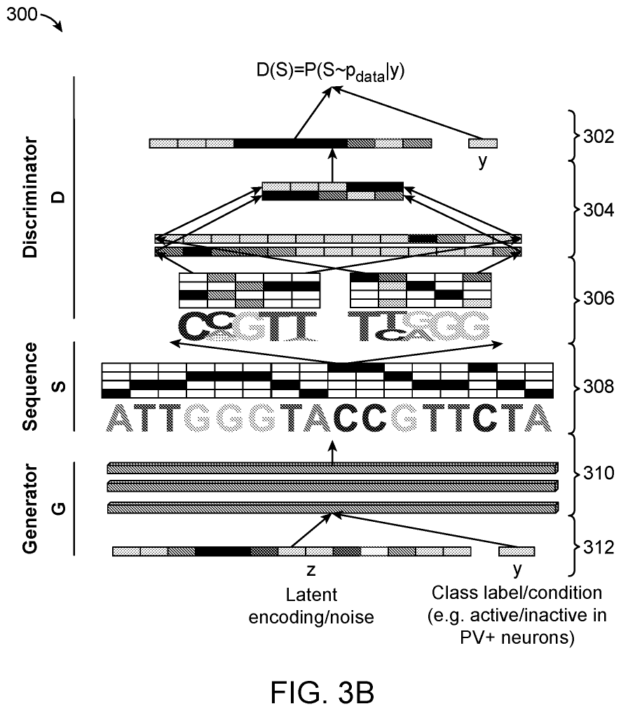

FIG. 3A shows an example process 350 for training and executing the machine learning model to generate synthetic genetic sequences based on the outcomes of the prediction of process 100, previously described. The results from the data processing system process 100 can guide a user to synthesize candidate REs and package them into an engineered adeno-associated virus (AAV) along with a transgene to express in the target cells. Transgenes can be a reporter gene, such as GFP, to validate experiments. SNAIL can also work with other transgenes, including ones that control neuronal firing or ones that report neuronal activation.

The process 350 includes receiving (352) training data associating at least one feature of a genetic sequence of the at least one cell type with expression of a hallmark of the at least one cell type. The process 350 includes training (354), based on the training data, a model configured to generate data representing a synthetic genetic sequence. The process 350 includes receiving (356) input data including the at least one feature of the genetic sequence. The process 350 includes generating (358) the synthetic genetic sequence in response to receiving the input data comprising the at least one feature.

In some implementations, the machine learning model includes a GAN. The GAN generally includes a generator network configured to receive latent random noise data as the input data and generate the synthetic genetic sequence and a discriminator network configured to generate a probability value representing whether the input sequence is drawn from the synthetic genetic sequence from the generator network or from a distribution of natural genetic sequences. Turning briefly to FIG. 3B, an example machine learning model 300 is shown for generating data representing a synthetic genetic sequence configured for labeling at least one cell type by causing expression of a marker in the at least one cell type.

Generally, the machine learning model 300 is configured to generate data that models the SNAIL constructs (e.g., viral probes) discussed throughout this specification. The model 300 is a particular model including a DCGAN that has a generator G, a sequence S, and a discriminator D. However, as previously described, while the DCGAN is shown for illustrative purposes, other machine learning models are possible, such as support vector machines (SVNs), neural networks (NN) such as CNNs, and so forth. The models for generating regulatory sequences are not specific for SVM and CNNs, but also can utilize generative components to the CNNs that relate genomic sequences to regulatory activity and use them to construct synthetic enhancer sequences. Machine learning models, such as model 300, are configured to construct synthetic enhancer sequences that are computationally optimized to drive cell type-specific labeling when used in an AAV construct, more so than the endogenous genomic sequences that were used to train the CNN classifiers.

Returning to FIG. 3A, in some implementations, the machine learning model is configured for generating a nucleic acid sequence comprising the synthetic genetic sequence. In this example, the nucleic acid sequence comprises the synthetic genetic sequence operably linked to a nucleotide sequence encoding a marker. For example, the marker can include a Sun1GFP fusion polypeptide. In another example, the nucleic acid sequence further comprises a virus nucleic acid sequence. Here, the virus can include an adeno-associated virus (AAV).

The machine learning model is configured for receiving results data representing a delivery of a nucleic acid including the synthetic genetic sequence to an organism. The results data represents a successful labeling of the at least one cell type or an unsuccessful labeling of the at least one cell type. To train the model, the model is updated using the results data. The model is then configured to generate an updated synthetic genetic sequence based on the updated model. Updating the model can also be done by receiving training data including the hallmark representing marker positive results marker negative results, or both. The at least one feature corresponding to the marker positive result or to the marker negative result is extracted. The at least one feature is then added to the model. In some implementations, the feature is a set of k-mer or gapped k-mer counts. Extracting the at least one feature can include scanning the sequence to determine the set of k-mer counts that form the sequence.

Other models besides the GAN of FIG. 3B can be used. For example, a support vector machine includes a feature space a support vector representing a classification border in the feature space. The model is executed to add, to the support vector of the support vector machine, a given set of k-mer counts within a predefined distance of the classification border in the feature space of the support vector machine.

In another example, the machine learning model includes a neural network. For example, the machine learning model can include a convolutional neural network. The neural network is loaded with one or more weight values (also called activation values) each associated with a feature of the synthetic genetic sequence. For example, a feature of the neural network can include a set of k-mer counts of the genetic sequence. In some implementations, the feature represents a transcription factor binding motif.

In any of the machine learning models described in this specification, the synthetic genetic sequence that is generated from the model is configured to distinguish between cell types. The particular cell types are based on the training data used. For example, the genetic sequence that is generated from the model can be configured to distinguish between parvalbumin positive (PV+) and parvalbumin negative (PV−) cells (e.g., the generated sequence can lead to expression in at least 70%, at least 80%, at least 90%, at least 95%, or at least 99% of all PV+ neurons that are transduced by the virus and have the chance to express the machinery, but would not lead to expression in at least 70%, at least 80%, at least 90%, at least 95%, or at least 99% of PV-cells). For example, the genetic sequence that is generated from the model can be configured to distinguish between PV+ and excitatory (EXC) neurons (e.g., the generated sequence can lead to expression in at least 70%, at least 80%, at least 90%, at least 95%, or at least 99% of all PV+ neurons that are transduced by the virus and have the chance to express the machinery, but would not lead to expression in at least 70%, at least 80%, at least 90%, at least 95%, or at least 99% of EXC neurons). For example, the generated genetic sequence that is generated from the model can be configured to distinguish between PV+ and vasoactive intestinal peptide-expressing (VIP+) neurons (e.g., the generated sequence can lead to expression in at least 70%, at least 80%, at least 90%, at least 95%, or at least 99% of all PV+ neurons that are transduced by the virus and have the chance to express the machinery, but would not lead to expression in at least 70%, at least 80%, at least 90%, at least 95%, or at least 99% of VIP+ neurons). However, other cell types can be distinguished by the generated genetic sequence based on the training data used. These examples are non-exhaustive and illustrate possible examples from hundreds or thousands of cell types. The regulatory sequence features learned may extend beyond cell types as well as to cellular states, such as neurons that in the process or responding to neural activity or cells that are responding to DNA damage.

As shown in FIG. 3B, the DCGAN for labeling at least one cell type by causing expression of a marker in the at least one cell type includes two neural network modules, a generator network G and a discriminator network D. The generator G receives latent random noise input (z) and outputs a synthetic sequence. The discriminator network is configured to generate probability value representing a probability that an input sequence is drawn from a distribution of real sequences. The parameters of both G and D can be tuned together using alternating phases of training with real sequences from existing data sources and synthetic sequences generated from G. As the models are trained, G generally generates synthetic sequences that are closer to the real data distribution than before. The discriminator is more likely to classify these generate sequences as genuine.

The generator is training G to generate realistic sequence data. For example, a GAN could be applied to generate synthetic enhancers that are likely highly active in PV+ or PV− neurons by training on real data drawn from endogenous genomic sequences underlying previously profiled open chromatin regions in PV+ and PV− neurons. Further, to optimize specific properties of the sequence, a conditional GAN can be used where an additional class label (y) is input to both G and D. For example, to generate sequences specific to PV+ neurons relative to PV− neurons, a GAN could be trained on real sequences active in both PV+ and PV−neurons, but the generation can be forced to be specific to PV+ neurons by setting y to be 1 for all sequences active in PV+ neurons and 0 for all sequences active in PV− neurons. This enables discriminative generation of sequences, where the optimized property is enhancer activity in a cell type relative to surrounding or background cell types.

Generally, using the GAN includes training the generator network and the discriminator network of the GAN by alternating the input data between synthetic sequence derived from the latent noise data input to the generator network and natural genetic sequence data. The GAN receives input data representing endogenous genomic sequences underlying previously profiled open chromatin regions of at least two cell types and then updates the discriminator network for distinguishing between the at least two cell types. In an example, the at least two cell types include parvalbumin positive (PV+) and parvalbumin negative (PV−) neurons; however, other cell types can be used, and are described throughout this specification. In this example, the discriminator network is configured to distinguish between the PV+ and PV− neurons based on the input data, but other cell types can be used as well, such as excitatory (EXC) neurons, vasoactive intestinal peptide-expressing (VIP+) neurons, and so forth.

The GAN is configured for optimizing a feature of the synthetic genetic sequence by applying a class label to both the generator network and the discriminator network. The class label is configured to force a first probability value for a first input type and a second probability value for a second input type that is different from the first input type. In some implementations, the feature represents an enhancer of an activity in a cell type.

The model 300 is configured to receive (312) the noise and condition input together to generate a compressed representation of the output sequence. The compressed representation is unpacked (310) using a deconvolution process. Fully connected layers can be used in the model 300 to generate a synthetic sequence. The model 300 is configured to generate (308) sequence binary encoding to input to the discriminator. The discriminator is configured to determine whether the sequence it receives is a synthetic sequence or a naturally occurring sequence. The discriminator includes a one dimensional (1D) convolution layer configured to scan (306) the received sequences for patterns. Max pooling can be used (304) to down-sample the input sequence. This convolution and max pooling process can be iterated as needed. The class label (e.g., condition) and compressed output from multiple fully connected layers are output (302) into an output unit.

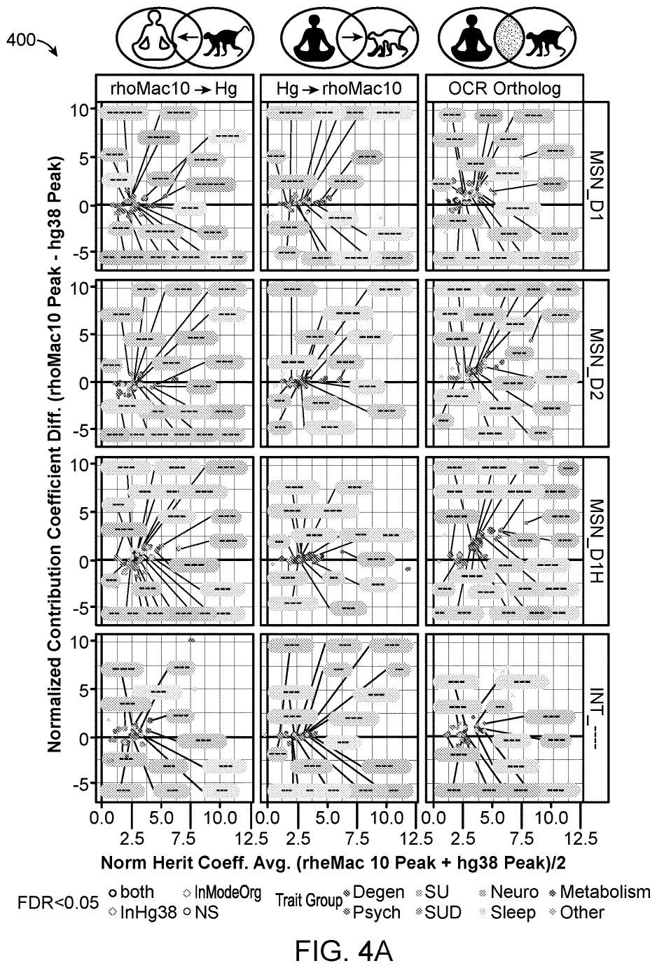

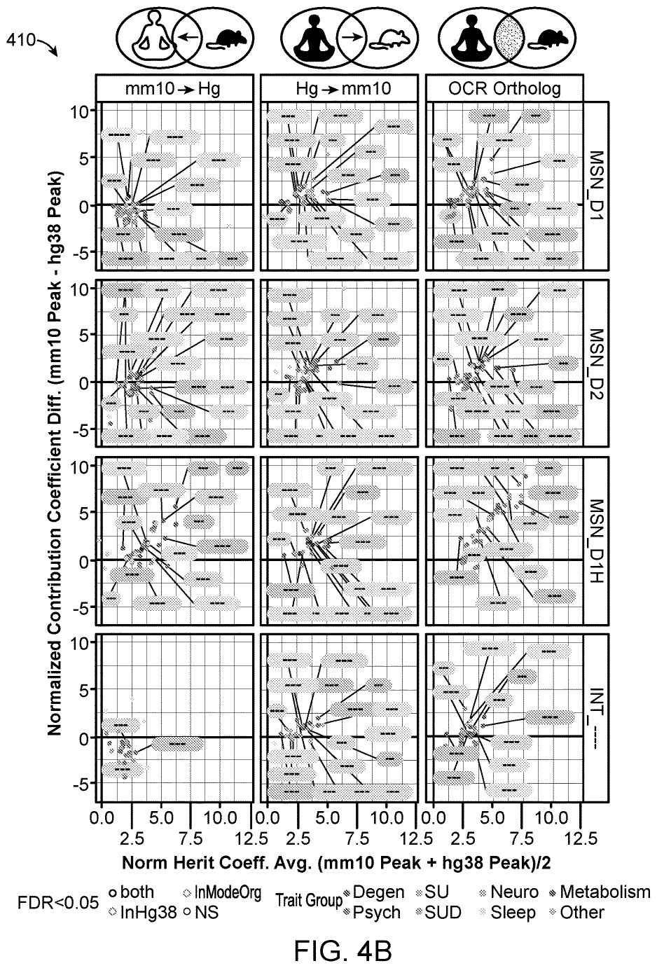

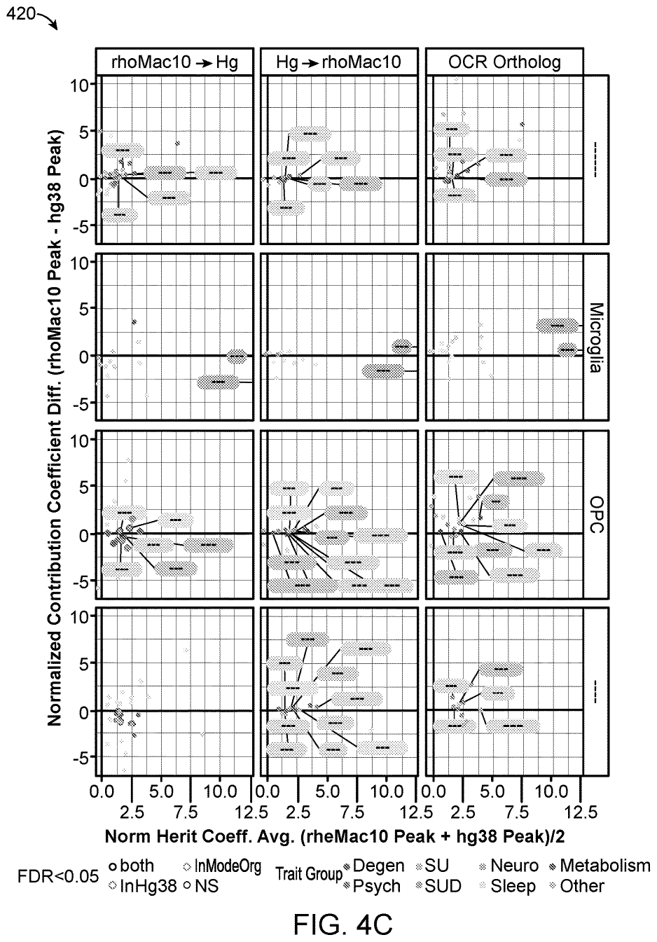

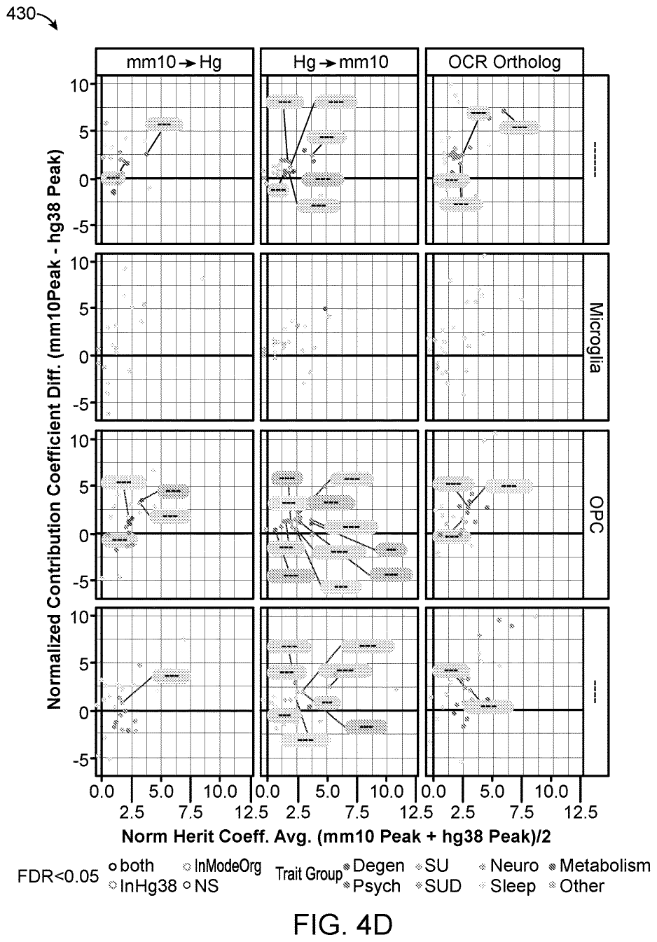

FIGS. 4A-4D show examples of pairwise human-model species caudate cell type OCR comparisons that demonstrate complex traits enriched within conserved open chromatin in the human genome. To validate the concept that cell type-specific OCRs are more likely to be functional, enrichment of candidate OCRs for genetic variants from genome-wide association studies is performed. A case study of the method 100 is performed using dorsal striatum in relation to neural and psychiatric traits. The data processing system verified a strong enrichment of disease-associated genetic variants in the enhancers that were conserved in a specific neuron subtypes between human and macaque cell data, shown in panels 400 of FIG. 4A. The data processing system verified a strong enrichment of disease-associated genetic variants in the enhancers that were conserved in a specific neuron subtypes between human and mouse cell data, shown in panels 410 of FIG. 4B.

The pairwise human-model species caudate cell type OCR comparisons demonstrate complex traits enriched within conserved open chromatin in the human genome. Conditional independence (T*, normalized heritability coefficient) LD score regression (LDSC) analyses of caudate cell type open chromatin regions (OCRs) using human, rhesus macaque, and mouse open chromatin. Each point is a GWAS colored by the trait group (Data S1). The r* was estimated for each GWAS in 7 OCR sets for each cell type: 0) human OCRs; 1) rhesus macaque OCRs mapped to human genome, rheMac10→Hg; 2) human OCRs that can be mapped to rhesus macaque, Hg→rheMac10; 3) overlap of human and rhesus OCRs and OCR orthologs 4) mouse OCRs that can be mapped to human, mm10→Hg; 5) human OCRs that can be mapped to mouse, Hg→mm10; and 6) overlap of human and mouse OCRs and OCR ortholog. The τ* for OCR sets mappable or open chromatin conserved in other species (1-6) are compared to human OCRs (0) using a mean-difference plot where the average τ* are plotted on the x-axis (normalized heritability coefficient average) and the change in τ* with mappability or orthology in model species are plotted on the y-axis (normalized heritability coefficient difference). Points above the y=0 line demonstrate higher τ* with genome or open chromatin conservation information above human cell type OCRs alone. Significant τ* LDSC estimates are outlined with red for significance in human OCRs (inHg38), significant in Model Species (inModelOrg, 1-6), or both (FDR<0.05, computed over all 8 cell types, 64 traits, 7° C. R set comparisons). Traits that does not have significant τ* estimates are plotted with transparency.

For panels 400, 410, neuronal cell type comparisons are shown including: MSN_D1, MSN_D2, MSN_SN, and INT_Pvalb. For panels 420, 430, glial cell type comparisons are shown: Astro, Microglia, OPC, and Oligo.

These conserved enhancers were compared to OCRs that were identified macaque mapped to human cell types, shown in panels 420 of FIG. 4C. These conserved enhancers were compared to OCRs that were identified in human and could map to macaque, shown in panels 430 of FIG. 4D. The conserved OCRs shows substantially greater enrichment for genetic variants associated with neuropsychiatric traits.

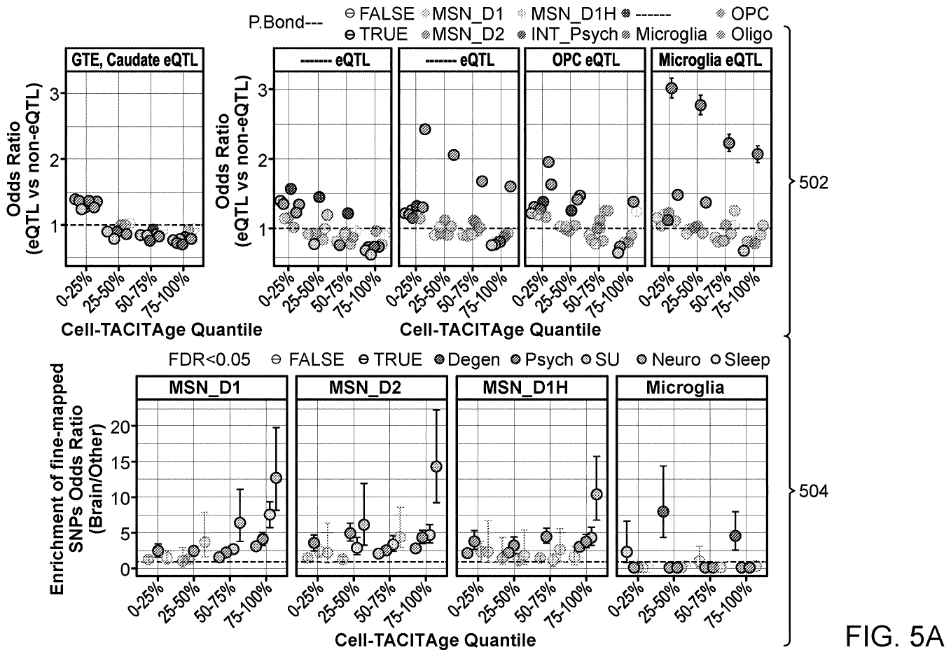

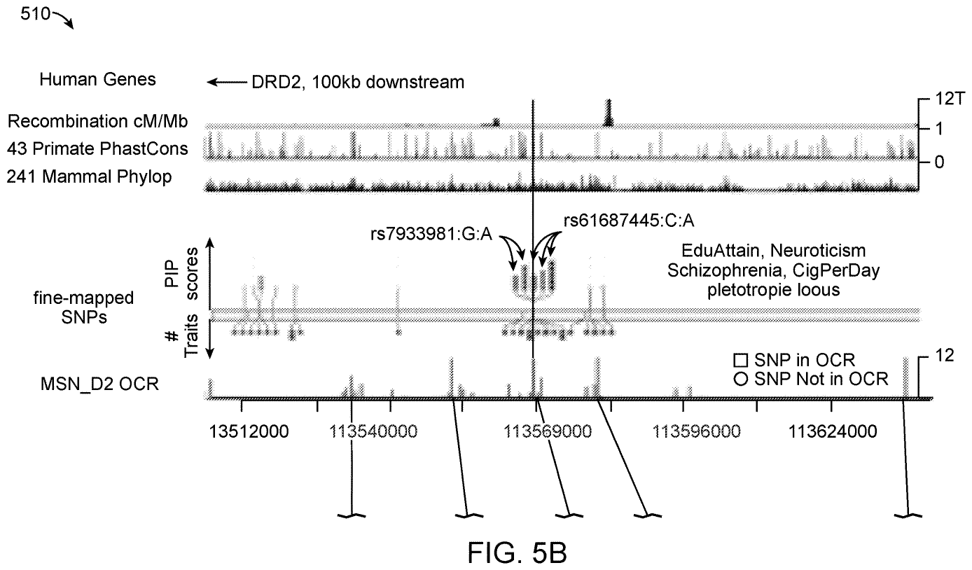

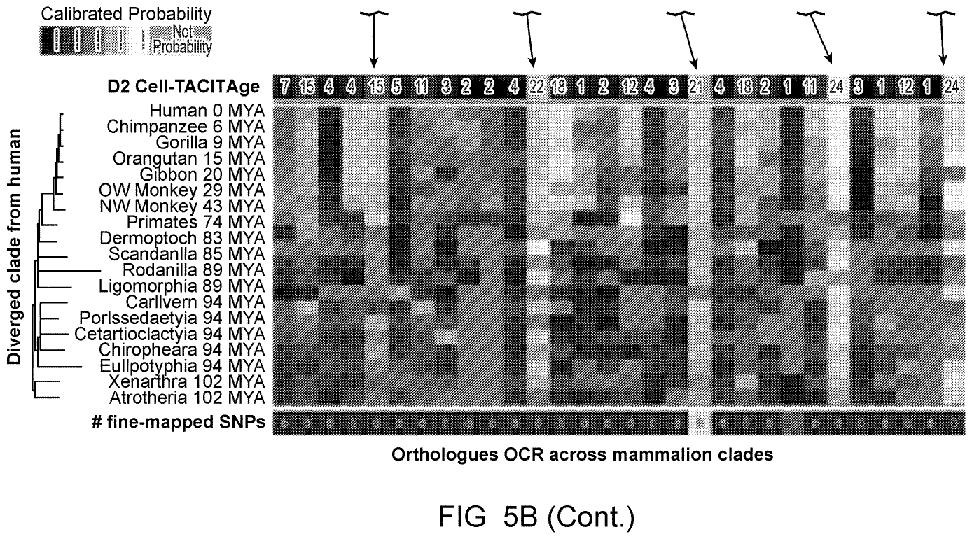

FIGS. 5A-5B show a process for annotating cell type eQTLs and fine-mapped SNPs with Cell-TACIT age estimates conserved cell type regulatory activity across mammalian species. To validate the concept that cell type-specific OCRs conserved in predicted open chromatin are more likely to be functional, the data processing system uses enrichment of candidate OCRs for genetic variants from genome-wide association studies and mouse reporter assay experiments. For each human OCR in each cell type of striatum, the data processing system determine a Cell-TACIT age, which corresponds to the evolutionary distance at which the orthologous regions of open chromatin are predicted to be active. The Cell-TACIT age in specific cell subtypes showed significant associations with cell type-specific eQTLS, as shown in panels 500 and 502 if FIG. 5A. Specifically, panel 500 includes a strip-chart plotting the odds ratio (±standard error of the mean) that significant eQTLs from bulk caudate nucleus RNA-seq are enriched in caudate cell type open chromatin regions stratified by the Cell-TACIT Age. The top age quantiles are regions predicted to be “older” and retain activity in distant placental mammals. Each point is colored by the corresponding Cell-TACIT model. Solid colored points indicate significant enrichment at Bonferroni corrected P-value <0.05. Panels 502 show enrichment strip-chart eQTLs from single-cell prefrontal cortex cell types with Cell-TACIT Age quartiles. These prefrontal cortex cell types have one-to-one correspondence with a Cell-TACIT cell type model.

Higher cell-TACIT ages, indicating greater levels of conservation, were associated with substantially stronger cell type-specific enrichments for psychiatric and other neural traits, shown in panels 506 of FIG. 5A. Specifically, panels 506 include enrichment strip-charts for fine-mapped neuropsychiatric trait SNPs with PIP >0.10 in Cell-TACIT age quartiles for the top caudate neural cell types. Each point is colored by enrichment for SNPs fine-mapped in neurodegenerative traits (Degen), psychiatric traits (Psych), substance use traits (SU), neurological traits (Neuro), or sleep traits (Sleep). Solid points are significant enrichment at FDR corrected P-value <0.05.

FIG. 5B shows an example locus and fine-mapped SNP demonstrates how cell-TACIT age is calculated. Specifically, a ILocus plot 100 kb downstream from the DRD2 gene with SNPs and Cell-TACIT annotations for D2 MSN models identifies candidate regulatory elements with fine-mapped SNPs from education attainment, neuroticism, schizophrenia, and cigarettes per day GWAS. Cell-TACIT Age (top row) heatmap for D2 MSNs summarizes the predicted activity of each open chromatin region in the track plot across increasingly distant clades of placental mammals (middle rows). The phylogenetic tree annotates the millions of years ago (MYA) that the common ancestor of each group of placental mammals and humans has diverged. Two D2 MSN open chromatin regions contain fine-mapped SNPs in this locus (bottom row).

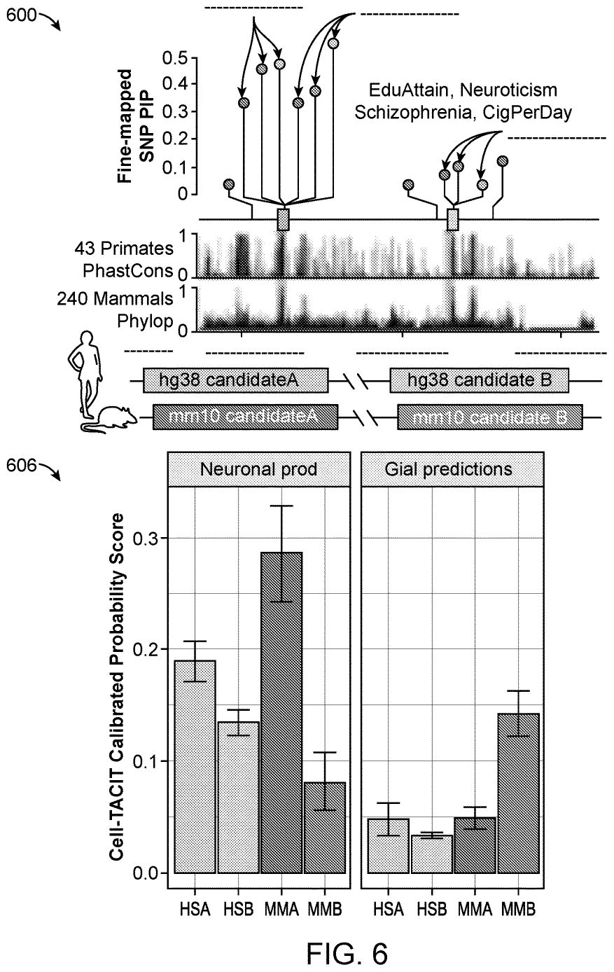

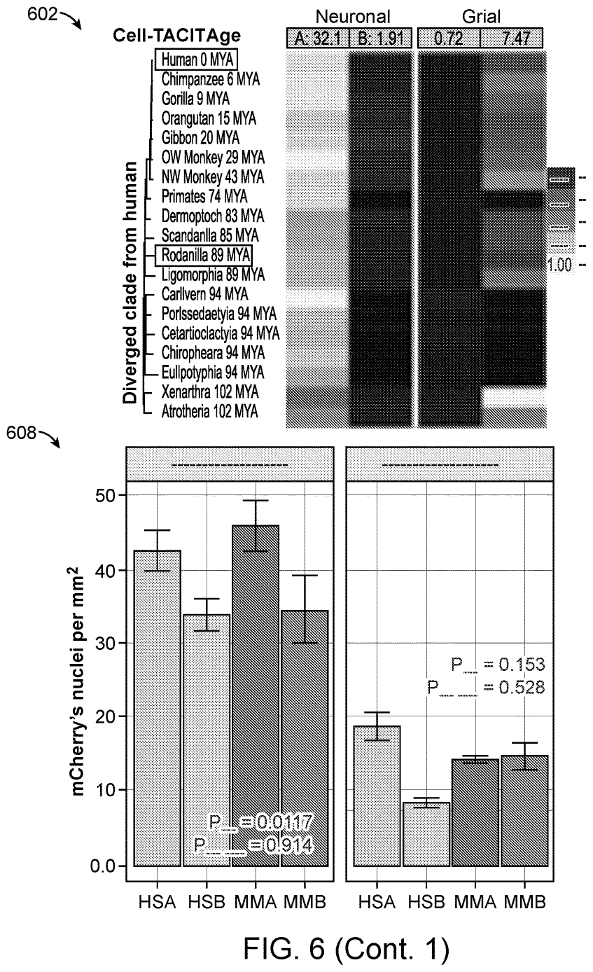

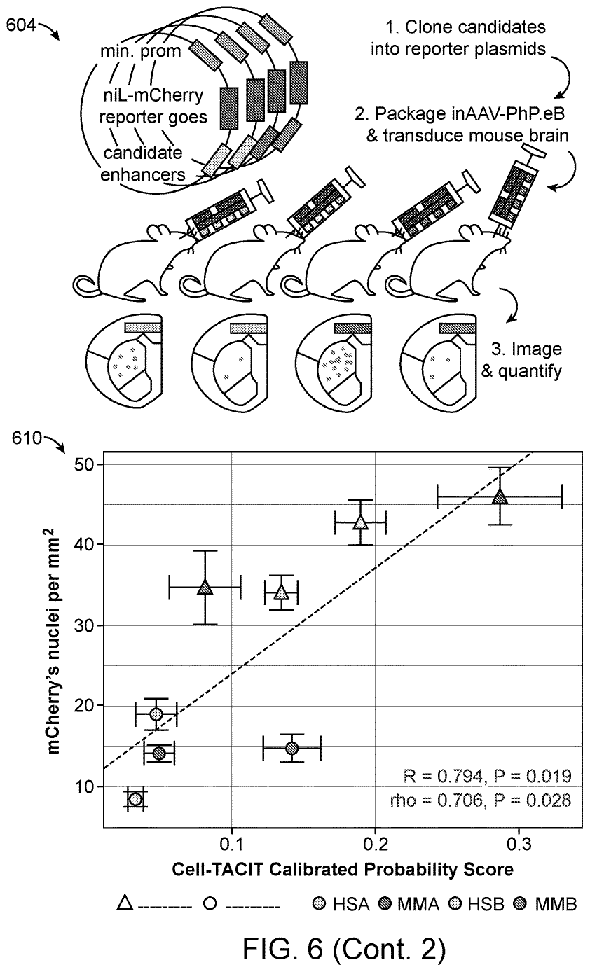

FIG. 6 shows example data 600 of an experimental validation of candidate enhancers in a reporter assay. Data 602 shows a position of two candidate enhancers at the DRD2 locus (bottom) is shown along with annotation of SNPs implicated by GWAS (top), and nucleotide-based conservation scores (middle). Data 602 shows orthologs of the candidate enhancers shows that candidate A is predicted to be highly conserved in its neuronal function while enhancer B is predicted to be weakly conserved in its glial function. Data 604 shows that candidate enhancers are cloned into constructs in which they will drive the mCherry reporter. Data 606 shows the activity score predicted by the machine learning model for each cell type (neurons vs. glial cells) is plotted. Errors bars are calculated based on the weighted average of different cell subtypes that make up the population. Data 608 shows a measured reporter assay activity based on the proportion of mCherry positive nuclei is plotted for n=3 mice (2 male, 1 female). P-values are calculated based on a t-test. Data 610 shows the proportion of mCherry positive nuclei is plotted against the predicted activity based on the machine learning model.

The evolutionary analysis identified dozens of open chromatin regions implicated in GWAS and predicted to be conserved open chromatin regions in specific neural cell types. The data processing system validates the activity of these enhancers in low throughput reporter assays in the mouse brain. Two candidate open chromatin regions were chosen at the DRD2 locus, which has been implicated in smoking behavior (600, 602). Both human and mouse orthologs of these candidate enhancers were cloned into a plasmid vector in which it would drive the expression of mCherry (604). The vectors were packaged into AAV.PHP.eb for system transduction in mouse brain. The data processing system measured the fluorescence of the reporters in the striatum and simultaneously labeled neuron (NeuN+) and glia (NeuN−). There is a strong correspondence between the cell type-specific activity of the enhancer predicted by machine learning13 and the measured fluorescence within that cell type, shown in data 606, 608, and 610. These results suggest that that machine learning models are able to predict in vivo cell type-specificity. These results are consistent with similar approaches that we have used to predict the cell type-specific of candidate regulatory elements.

The cell type specific tools enable scientists to study the causal relationships between these cell types and behavior. Moreover, because the molecular definitions are derived from nonhuman primate (NHP), they have a high degree of homology with human cell types, and this greatly increases the translational potential. These cell type specific enhancers could be used in concert with Designer Receptors Exclusively Activated by Designer Drugs (DREADDs), to selective activate or inhibit neuron subtypes underlying a variety of neurological and psychiatric disorders including Parkinson's and major depressive disorder.

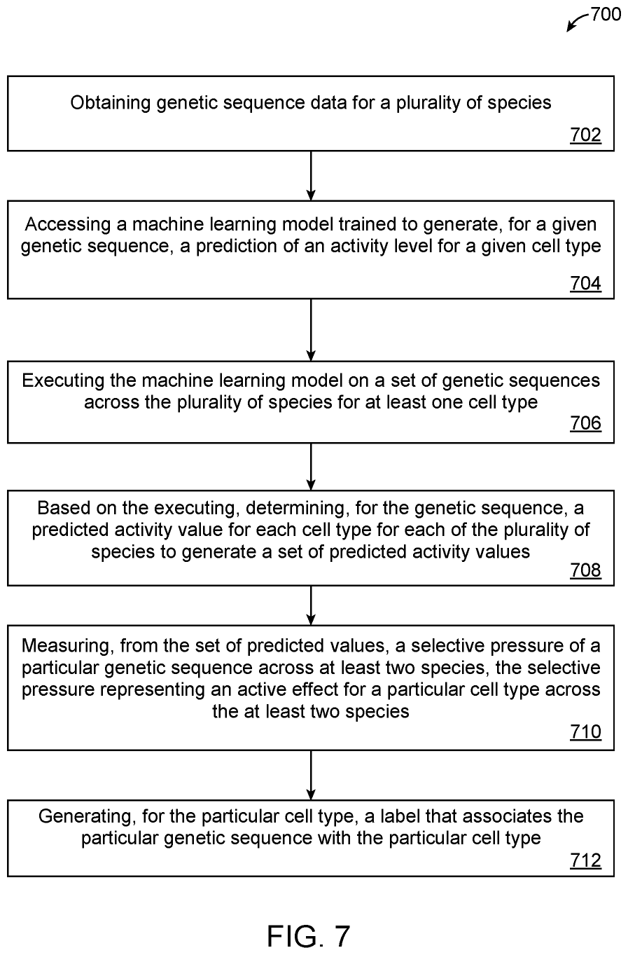

FIG. 7 shows an example process 700 for interpreting genetic variants of complex neurological disorder traits with machine learning models inferring conserved functional genomes of caudate cell types across species. In some implementations, the process 700 is performed by a data processing system (such as data processing system 800 of FIG. 8) including one or more processors. The process 700 includes obtaining (702) genetic sequence data for a plurality of species. The process 700 includes accessing (704) a machine learning model trained to generate, for a given genetic sequence, a prediction of an activity level for a given cell type. The process 700 includes executing (706) the machine learning model on a set of genetic sequences across the plurality of species for at least one cell type. The process 700 includes, based on the executing, determining (708), for the genetic sequence, a predicted activity value for each cell type for each of the plurality of species to generate a set of predicted activity values. The process 700 includes measuring (710), from the set of predicted values, a selective pressure of a particular genetic sequence across at least two species, the selective pressure representing an active effect for a particular cell type across the at least two species. The process 700 includes generating (712), for the particular cell type, a label that associates the particular genetic sequence with the particular cell type.

In some implementations, the process 700 includes testing the particular gene sequence to validate the predicted activity value of the genetic sequence for the particular cell type for a species of the at least two species. In some implementations, the testing includes introducing a gene sequence into a genome for the particular cell type; and measuring a gene expression for the particular cell type.

In some implementations, the machine learning model is trained by performing operations comprising training the machine learning model with input data labeled by features of a given genetic sequence correlating to a similar enhancer behavior for a cell type across two or more types of species; training the machine learning model with input data labeled by features of the given genetic sequence correlating to different enhancer behavior for the cell type across the two or more types of species; and training the machine learning model with input data where the labels are inferred across orthologous genome sequences and cell populations.

In some implementations, the features comprise identified sets of one or more nucleotides in the genetic sequence.

In some implementations, the process 700 includes determining a set of gene sequences that are associated with the particular cell type; and training, using the set of gene sequences, a second machine learning model to generate a given synthetic gene sequence as an enhancer sequence for the particular cell type.

In some implementations, the process 700 includes generating, using the second machine learning model, a synthetic gene sequence that acts as an enhancer sequence for the particular cell type.

In some implementations, a first species of the plurality of species is a human species, and wherein a second species of the plurality is a rodent species.

In some implementations, the genetic sequence includes an enhancer sequence.

In some implementations, the genetic sequence includes an orthologous sequence across the at least two species.

In some implementations, the genetic sequence data comprises chromatin data.

In some implementations, the plurality of species includes at least 100 species types, and wherein the at least one cell type comprises at least 10 cell types.

### Definitions

Enhancers include deoxyribonucleic acid (DNA)-regulatory elements that activate transcription of a gene or genes to higher levels than would be the case in their absence. Genome profiling has revealed that general transcription factors (GTFs) and ribonucleic acid (RNA) polymerase II (Pol II) are recruited to enhancers. Enhancers serve as centers for the assembly of the pre-initiation complex (PIC).

Chromatin data refers to data describing a mixture of DNA and proteins that form the chromosomes found in the cells of humans and other higher organisms. Chromatin packages DNA into a unit capable of fitting within the tight space of a nucleus. There are two forms of chromatin-euchromatin and heterochromatin. Euchromatin consists of DNA segments involved in replication and transcription.

Epigenomics includes determining heritable marks that are materialized by chemical modification of the nucleotide bases and histones. Histones are a major type of chromosomal proteins, which are compactly wrapped around by genomic DNAs to form chromatins, to reduce chromosomal volume and strengthen the structure. The chemical modifications alongside the DNA sequences carry heritable information, passed down from cells to their offspring, by the completion of either mitotic or meiotic cell cycles. Modifications may affect the binding of transcription factors to the transcription elements, thereby changing the expression of genes.

The terms “nucleic acid” and “polynucleotide” can be used interchangeably, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

As used herein, “isolated,” when in reference to a nucleic acid, refers to a nucleic acid that is separated from other nucleic acids that are present in a genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as DNA that is incorporated into a vector, an autonomously replicating plasmid, a virus (e.g., a pararetrovirus, a retrovirus, lentivirus, adenovirus, or herpes virus), or the genomic DNA of a prokaryote or eukaryote. In addition, an isolated nucleic acid can include a recombinant nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made by, for example, chemical synthesis or polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be obtained by mutagenesis. For example, a naturally occurring nucleic acid sequence can be mutated using standard techniques, including oligonucleotide-directed mutagenesis and site-directed mutagenesis through PCR. See, Short Protocols in Molecular Biology, Chapter 8, Green Publishing Associates and John Wiley & Sons, edited by Ausubel et al., 1992.

Recombinant nucleic acid constructs (e.g., vectors) containing sequences encoding the tagged Sun1 fusion polypeptides also are provided herein. A “vector” is a replicon, such as a plasmid, phage, or cosmid, into which another DNA segment may be inserted so as to bring about the replication of the inserted segment. Vector backbones include, for example, plasmids, viruses, artificial chromosomes, bacterial artificial chromosomes (BACs), yeast artificial chromosomes (YACs), and phage artificial chromosomes (PACs), as well as RNA vectors, and linear or circular DNA or RNA molecules that include chromosomal, non-chromosomal, semi-synthetic, or synthetic nucleic acids. Vectors include those capable of autonomous replication (episomal vectors) and/or expression of nucleic acids to which they are linked (expression vectors). Generally, a vector is capable of replication when associated with the proper control elements. The term “vector” includes cloning and expression vectors, as well as viral vectors and integrating vectors. An “expression vector” is a vector that includes one or more expression control sequences to control and regulate the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available.

Viral vectors include, without limitation, retrovirus, adenovirus, parvovirus (e.g., adeno associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picor-navirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, “Retroviridae: The viruses and their replication,” in Fundamental Virology, Third Edition, B. N. Fields, et al., eds., Lippincott-Raven Publishers, Philadelphia, 1996).

The terms “regulatory region,” “control element,” and “expression control sequence” refer to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, and other regulatory regions that can reside within coding sequences, such as secretory signals, Nuclear Localization Sequences (NLS) and protease cleavage sites.

As used herein, “operably linked” means incorporated into a genetic construct so that expression control sequences effectively control expression of a coding sequence of interest. A coding sequence is “operably linked” and “under the control” of expression control sequences in a cell when RNA polymerase is able to transcribe the coding sequence into RNA, which if an mRNA, then can be translated into the protein encoded by the coding sequence. Thus, a regulatory region can modulate, e.g., regulate, facilitate or drive, transcription in the plant cell, plant, or plant tissue in which it is desired to express a modified target nucleic acid.

A promoter is an expression control sequence composed of a region of a DNA molecule, typically (but not always) within 100 nucleotides upstream of the point at which transcription starts (generally near the initiation site for RNA polymerase II). Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically includes at least a core (basal) promoter. A promoter also may include at least one control element such as an upstream element. Such elements include upstream activation regions (UARs) and, optionally, other DNA sequences that affect transcription of a polynucleotide such as a synthetic upstream element.

The choice of promoters to be included depends upon several factors, including, but not limited to, efficiency, selectability, inducibility, desired expression level, and cell or tissue specificity. For example, tissue-, organ- and cell-specific promoters that confer transcription only or predominantly in a particular tissue, organ, and cell type, respectively, can be used. In some cases, a promoter that is active in a particular type of cell (e.g., a particular type of neuron) can be used. Such promoters- or other expression control sequences—can be identified using methods such as those described herein and can be used in the SNAIL constructs provided herein. Alternatively, constitutive promoters can promote transcription of an operably linked nucleic acid in essentially any tissue of an organism. Such promoters can be used in the cSNAIL constructs provided herein. Other classes of promoters include, without limitation, inducible promoters that confer transcription in response to external stimuli such as chemical agents, developmental stimuli, or environmental stimuli.

Generally, as previously discussed, machine learning models can be used for generating data representing a synthetic genetic sequence configured for labeling at least one cell type by causing expression of a marker in the at least one cell type. The models can include GANs, such as DCGANs, CNNs, support vector machines (SVMs), and so forth. The machine learning models are configured for generating the synthetic genetic sequences that are candidates for the SNAIL process (e.g., as viral probes).

FIG. 8 is a block diagram of an example computer system 800 used to provide computational functionalities associated with described algorithms, methods, functions, processes, flows, and procedures described in the present disclosure, according to some implementations of the present disclosure. The illustrated computer 802 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 802 can include input devices such as keypads, keyboards, and touch screens that can accept user information. Also, the computer 802 can include output devices that can convey information associated with the operation of the computer 802. The information can include digital data, visual data, audio information, or a combination of information. The information can be presented in a graphical user interface (UI) (or GUI).

A convolutional neural network (CNN) can be configured based on a presumption that inputs to the neural network correspond to image pixel data for an image or other data that includes features at multiple spatial locations. For example, sets of inputs can form a multi-dimensional data structure, such as a tensor, that represent features of chromatin data or cell type data (e.g., particular sequences, cell function, etc.). A convolutional layer of the convolutional neural network can process the inputs to transform features of the image that are represented by inputs of the data structure. For example, the inputs are processed by performing dot product operations using input data along a given dimension of the data structure and a set of parameters for the convolutional layer.

Performing computations for a convolutional layer can include applying one or more sets of kernels to portions of inputs in the data structure. The manner in which a system performs the computations can be based on specific properties for each layer of an example multi-layer neural network or deep neural network that supports deep neural net workloads. A deep neural network can include one or more convolutional towers (or layers) along with other computational layers. Convolutional layers of a CNN can have sets of artificial neurons that are arranged in three dimensions, a width dimension, a height dimension, and a depth dimension. The depth dimension corresponds to a third dimension of an input or activation volume and can represent respective sequences of a gene.

In general, layers of a CNN are configured to transform the three dimensional input volume (inputs) to a multi-dimensional output volume of neuron activations (activations). A convolutional layer of a neural network of the machine learning system 822 computes the output of neurons that may be connected to local regions in the input volume. Each neuron in the convolutional layer can be connected only to a local region in the input volume spatially, but to the full depth (e.g., all sequence features) of the input data. For a set of neurons at the convolutional layer, the layer computes a dot product between the parameters (weights) for the neurons and a certain region in the input volume to which the neurons are connected. This computation may result in a volume such as 32×32×12, where 12 corresponds to a number of kernels that are used for the computation. A neuron's connection to inputs of a region can have a spatial extent along the depth axis that is equal to the depth of the input volume. The spatial extent corresponds to spatial dimensions (e.g., x and y dimensions) of a kernel.

A set of kernels can have spatial characteristics that include a width and a height and that extends through a depth of the input volume. Each set of kernels for the layer is applied to one or more sets of inputs provided to the layer. That is, for each kernel or set of kernels, the machine learning system 822 can overlay the kernel, which can be represented multi-dimensionally, over a first portion of layer inputs (e.g., that form an input volume or input tensor), which can be represented multi-dimensionally.

The machine learning system 822 can then compute a dot product from the overlapped elements. For example, the machine learning system 822 can convolve (or slide) each kernel across the width and height of the input volume and compute dot products between the entries of the kernel and inputs for a position or region of the image. Each output value in a convolution output is the result of a dot product between a kernel and some set of inputs from an example input tensor. The dot product can result in a convolution output that corresponds to a single layer input, e.g., an activation element that has an upper-left position in the overlapped multi-dimensional space. As discussed above, a neuron of a convolutional layer can be connected to a region of the input volume that includes multiple inputs. The machine learning system 822 can convolve each kernel over each input of an input volume. The machine learning system 822 performs this convolution operation by, for example, moving (or sliding) each kernel over each input in the region.

The machine learning system 822 moves each kernel over inputs of the region based on a stride value for a given convolutional layer. For example, when the stride is set to 1, then the machine learning system 822 moves the kernels over the region one pixel (or input) at a time. Likewise, when the stride is 2, then the machine learning system 822 moves the kernels over the region two pixels at a time. Thus, kernels may be shifted based on a stride value for a layer and the machine learning system 822 can repeatedly perform this process until inputs for the region have a corresponding dot product. Related to the stride value is a skip value. The skip value can identify one or more sets of inputs (2×2), in a region of the input volume, that are skipped when inputs are loaded for processing at a neural network layer.

An example convolutional layer can have one or more control parameters for the layer that represent properties of the layer. For example, the control parameters can include a number of kernels, K, the spatial extent of the kernels, F, the stride (or skip), S, and the amount of zero padding, P. Numerical values for these parameters, the inputs to the layer, and the parameter values of the kernel for the layer shape the computations that occur at the layer and the size of the output volume for the layer.

The computations (e.g., dot product computations) for a convolutional layer, or other layers, of a neural network involve performing mathematical operations, e.g., multiplication and addition, using a computation unit of a hardware circuit of the machine learning system. The design of a hardware circuit can cause a system to be limited in its ability to fully utilize computing cells of the circuit when performing computations for layers of a neural network.

The computer 802 can serve in a role as a client, a network component, a server, a database, a persistency, or components of a computer system for performing the subject matter described in the present disclosure. The illustrated computer 802 is communicably coupled with a network 824. In some implementations, one or more components of the computer 802 can be configured to operate within different environments, including cloud-computing-based environments, local environments, global environments, and combinations of environments.

At a high level, the computer 802 is an electronic computing device operable to receive, transmit, process, store, and manage data and information associated with the described subject matter. According to some implementations, the computer 802 can also include, or be communicably coupled with, an application server, an email server, a web server, a caching server, a streaming data server, or a combination of servers.

The computer 802 can receive requests over network 824 from a client application (for example, executing on another computer 802). The computer 802 can respond to the received requests by processing the received requests using software applications. Requests can also be sent to the computer 802 from internal users (for example, from a command console), external (or third) parties, automated applications, entities, individuals, systems, and computers.

Each of the components of the computer 802 can communicate using a system bus 804. In some implementations, any or all of the components of the computer 802, including hardware or software components, can interface with each other or the interface 806 (or a combination of both), over the system bus 804. Interfaces can use an application programming interface (API) 814, a service layer 816, or a combination of the API 814 and service layer 816. The API 814 can include specifications for routines, data structures, and object classes. The API 814 can be either computer-language independent or dependent. The API 814 can refer to a complete interface, a single function, or a set of APIs.

The service layer 816 can provide software services to the computer 802 and other components (whether illustrated or not) that are communicably coupled to the computer 802. The functionality of the computer 802 can be accessible for all service consumers using this service layer. Software services, such as those provided by the service layer 816, can provide reusable, defined functionalities through a defined interface. For example, the interface can be software written in JAVA, C++, or a language providing data in extensible markup language (XML) format. While illustrated as an integrated component of the computer 802, in alternative implementations, the API 814 or the service layer 816 can be stand-alone components in relation to other components of the computer 802 and other components communicably coupled to the computer 802. Moreover, any or all parts of the API 814 or the service layer 816 can be implemented as child or sub-modules of another software module, enterprise application, or hardware module without departing from the scope of the present disclosure.

The computer 802 includes an interface 806. Although illustrated as a single interface 806 in FIG. 8, two or more interfaces 806 can be used according to implementations of the computer 802 and the described functionality. The interface 806 can be used by the computer 802 for communicating with other systems that are connected to the network 824 (whether illustrated or not) in a distributed environment. Generally, the interface 806 can include, or be implemented using, logic encoded in software or hardware (or a combination of software and hardware) operable to communicate with the network 824. More specifically, the interface 806 can include software supporting one or more communication protocols associated with communications. As such, the network 824 or the interface's hardware can be operable to communicate physical signals within and outside of the illustrated computer 802.

The computer 802 includes a processor 808. Although illustrated as a single processor 808 in FIG. 8, two or more processors 808 can be used according to implementations of the computer 802 and the described functionality. Generally, the processor 808 can execute instructions and can manipulate data to perform the operations of the computer 802, including operations using algorithms, methods, functions, processes, flows, and procedures as described in the present disclosure.

The computer 802 also includes a database 820 that can hold data (such as chromatin data or cell data 820) for the computer 802 and other components connected to the network 824 (whether illustrated or not). For example, database 820 can be in-memory or a database storing data consistent with the present disclosure. In some implementations, database 820 can be a combination of two or more different database types (for example, hybrid in-memory and conventional databases) according to implementations of the computer 802 and the described functionality. Although illustrated as a single database 820 in FIG. 8, two or more databases (of the same, different, or combination of types) can be used according to implementations of the computer 802 and the described functionality. While database 820 is illustrated as an internal component of the computer 802, in alternative implementations, database 820 can be external to the computer 802.

The computer 802 also includes a memory 810 that can hold data for the computer 802 or a combination of components connected to the network 824 (whether illustrated or not). Memory 810 can store any data consistent with the present disclosure. In some implementations, memory 810 can be a combination of two or more different types of memory (for example, a combination of semiconductor and magnetic storage) according to implementations of the computer 802 and the described functionality. Although illustrated as a single memory 810 in FIG. 8, two or more memories 810 (of the same, different, or combination of types) can be used according to implementations of the computer 802 and the described functionality. While memory 810 is illustrated as an internal component of the computer 802, in alternative implementations, memory 810 can be external to the computer 802.

The application 812 can be an algorithmic software engine providing functionality according to implementations of the computer 802 and the described functionality. For example, application 812 can serve as one or more components, modules, or applications. Further, although illustrated as a single application 812, the application 812 can be implemented as multiple applications 818 on the computer 802. In addition, although illustrated as internal to the computer 802, in alternative implementations, the application 812 can be external to the computer 802.

The computer 802 can also include a power supply 818. The power supply 818 can include a rechargeable or non-rechargeable battery that can be configured to be either user—or non-user—replaceable. In some implementations, the power supply 818 can include power-conversion and management circuits, including recharging, standby, and power management functionalities. In some implementations, the power-supply 818 can include a power plug to allow the computer 802 to be plugged into a wall socket or a power source to, for example, power the computer 802 or recharge a rechargeable battery.

There can be any number of computers 802 associated with, or external to, a computer system including the computer 802, with each computer 802 communicating over network 824. Further, the terms “client,” “user,” and other appropriate terminology can be used interchangeably, as appropriate, without departing from the scope of the present disclosure. Moreover, the present disclosure contemplates that many users can use one computer 802 and one user can use multiple computers 802.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example LINUX, UNIX, WINDOWS, MAC OS, ANDROID, or IOS.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random-access memory (RAM), read only memory (ROM), phase change memory (PRAM), static random-access memory (SRAM), dynamic random-access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks.

Several implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Certain features that are described in this specification in the context of separate implementations can also be implemented, in combination, in a single implementation. Conversely, various features that are described in the context of a single implementation can also be implemented in multiple implementations, separately, or in any suitable sub-combination. The separation or integration of various system modules and components in the previously described implementations should not be understood as requiring such separation or integration in all implementations, and the described program components and systems can generally be integrated together in a single product or packaged into multiple products.

Accordingly, the previously described example implementations do not define or constrain the present disclosure. It will be understood that various modifications may be made without departing from the scope of the systems and methods described herein. Accordingly, other embodiments are within the scope of the following claims.

1. A method for labeling a cell type with a gene sequence, comprising:
obtaining genetic sequence data for a plurality of species;
accessing a machine learning model trained to generate, for a given genetic sequence, a prediction of an activity level for a given cell type;
executing the machine learning model on a set of genetic sequences across the plurality of species for at least one cell type;
based on the executing, determining, for the genetic sequence, a predicted activity value for the at least one cell type for each of the plurality of species to generate a set of predicted activity values;
measuring, based on the set of predicted activity values, a selective pressure of a particular genetic sequence associated with predicted activity values that exceed a threshold value across at least two species, the selective pressure representing an active effect for a particular cell type across the at least two species; and
generating, for the particular cell type, a label that associates the particular genetic sequence with the particular cell type.

2. The method of claim 1, further comprising:
testing the particular gene sequence to validate the predicted activity value of the genetic sequence for the particular cell type for a species of the at least two species, the testing comprising:
introducing a gene sequence into a genome for the particular cell type; and
measuring a gene expression for the particular cell type.

3. The method of claim 1, wherein the machine learning model is trained by performing operations comprising:
training the machine learning model with input data labeled by features of a given genetic sequence correlating to a similar enhancer behavior for a cell type across two or more types of species; and
training the machine learning model with input data labeled by features of the given genetic sequence correlating to different enhancer behavior for the cell type across the two or more types of species,
training the machine learning model with input data where the labels are inferred across orthologous genome sequences and cell populations.

4. The method of claim 3, wherein the features comprise identified sets of one or more nucleotides in the genetic sequence.

5. The method of claim 1, further comprising:
determining a set of gene sequences that are associated with the particular cell type; and
training, using the set of gene sequences, a second machine learning model to generate a given synthetic gene sequence as an enhancer sequence for the particular cell type.

6. The method of claim 5, further comprising:
generating, using the second machine learning model, a synthetic gene sequence that acts as an enhancer sequence for the particular cell type.

7. The method of claim 1, wherein a first species of the plurality of species is a human species, and wherein a second species of the plurality is a rodent species.

8. The method of claim 1, wherein the genetic sequence includes an enhancer sequence.

9. The method of claim 1, wherein the genetic sequence includes an orthologous sequence across the at least two species.

10. The method of claim 1, wherein the genetic sequence data comprises chromatin data.

11. The method of claim 1, wherein the plurality of species includes at least 100 species types, and wherein the at least one cell type comprises at least 10 cell types.

12. A data processing system for labeling a cell type with a gene sequence, the data processing system comprising:
at least one processor; and
a memory storing instructions, that, when executed by the at least one processor, cause the at least one processor to perform operations comprising:
receiving genetic sequence data for a plurality of species;
accessing a machine learning model trained to generate, for a given genetic sequence, a prediction of an activity level for a given cell type;
executing the machine learning model on a set of genetic sequences across the plurality of species for at least one cell type;
based on the executing, determining, for the genetic sequence, a predicted activity value for the at least one cell type for each of the plurality of species to generate a set of predicted activity values;
measuring, based on the set of predicted activity values, a selective pressure of a particular genetic sequence associated with predicted activity values that exceed a threshold value across at least two species, the selective pressure representing an active effect for a particular cell type across the at least two species; and
generating, for the particular cell type, a label that associates the particular genetic sequence with the particular cell type.

13. The data processing system of claim 12, wherein the machine learning model is trained by performing operations comprising:
training the machine learning model with input data labeled by features of a given genetic sequence correlating to a similar enhancer behavior for a cell type across two or more types of species; and
training the machine learning model with input data labeled by features of the given genetic sequence correlating to different enhancer behavior for the cell type across the two or more types of species.

14. The data processing system of claim 13, wherein the features comprise identified sets of one or more nucleotides in the genetic sequence.

15. The data processing system of claim 12, the operations further comprising:
determining a set of gene sequences that are associated with the particular cell type; and
training, using the set of gene sequences, a second machine learning model to generate a given synthetic gene sequence as an enhancer sequence for the particular cell type.

16. The data processing system of claim 15, further comprising:
generating, using the second machine learning model, a synthetic gene sequence that acts as an enhancer sequence for the particular cell type.

17. The data processing system of claim 12, wherein a first species of the plurality of species is a human species, and wherein a second species of the plurality is a rodent species.

18. The data processing system of claim 12, wherein the genetic sequence includes an enhancer sequence.

19. The data processing system of claim 12, wherein the genetic sequence includes an orthologous sequence across the at least two species.

20. (canceled)

21. The data processing system of claim 12, wherein the plurality of species includes at least 100 species types, and wherein the at least one cell type comprises at least 10 cell types.
